(19) United States
(12) Patent Application Publication
(10) Pub. No.: US 2021/0307972 A1
(43) Pub. Date: Oct. 7, 2021

(54) MOLDED SWAB HEAD

(71) Applicant: MAWI DNA TECHNOLOGIES LLC, Hayward, CA (US)

(72) Inventor: Bassam El-Fahmawi, Hayward, CA (US)

(21) Appl. No.: 17/221,697

(22) Filed: Apr. 2, 2021

Related U.S. Application Data
(60) Provisional application No. 63/004,727, filed on Apr. 3, 2020.
(60) Provisional application No. 63/010,623, filed on Apr. 15, 2020.
(60) Provisional application No. 63/014,677, filed on Apr. 23, 2020.
(60) Provisional application No. 63/019,188, filed on May 1, 2020.
(60) Provisional application No. 63/032,285, filed on May 29, 2020.

(51) Int. Cl. A61F 13/38; A61B 10/02

(52) U.S. Cl. CPC A61F 13/38; A61F 2013/51078; A61B 10/02; A61F 13/385

(57) ABSTRACT

A swab for collecting biological samples where the head portion and the handle portion of the swab are formed together from a single piece of material.

### CROSS-REFERENCE TO RELATED APPLICATION(S)

This application claims the benefit of and priority to U.S. Provisional Patent Application No. 63/004,727, filed on Apr. 3, 2020, U.S. Provisional Patent Application No. 63/010,623, filed on Apr. 15, 2020, U.S. Provisional Patent Application No. 63/014,677, filed on Apr. 23, 2020, U.S. Provisional Patent Application No. 63/019,188, filed on May 1, 2020, and U.S. Provisional Patent Application No. 63/032,285, filed on May 29, 2020, the entire contents of each of which are fully incorporated herein by reference.

### FIELD OF THE INVENTION

The embodiments described here relate to a swab for taking biological samples, and more particularly, to a swab having a swab head formed from a non-fibrous and non-foam material.

### BACKGROUND OF THE INVENTION

Typical biological sample swabs typically include a handle and a swab tip or head formed from a fibrous material such as cotton, rayon, and polyester or a foam-type material.

### SUMMARY

In one embodiment, a swab for collecting biological samples, the swab comprising a head portion, and a handle portion extending from the head portion to define an axis therethrough, and where the head portion and the handle portion are formed together from a single piece of material.

In another embodiment, a swab for collecting biological samples, the swab including a handle having a first end and a second end opposite the first end, a head coupled to the handle at the first end thereof. Where the head includes a base proximate the first end of the handle, a tip spaced a distance from the base opposite the handle, and a plurality of legs each extending between the base and the handle.

In another embodiment, a method of manufacturing a swab for collecting biological samples, where the swab includes a handle portion and a head portion. The method includes providing a mold defining a mold cavity, where the mold cavity at least partially defines both the head portion and the handle portion, injecting a first material into the mold cavity to produce both the head portion and the handle portion of the swab, and ejecting the resulting part from the mold cavity.

### BRIEF DESCRIPTION OF THE DRAWINGS

FIG. 1 is a perspective view of the swab of the present invention.

FIG. 2 is a side view of the swab of FIG. 1.

FIG. 3 is a side view of a head portion of the swab of FIG. 1.

FIG. 4 is a side view of another embodiment of a head portion of the swab of FIG. 1.

FIG. 5 is a side view of another embodiment of a head portion of the swab of FIG. 1.

FIG. 6 is a top view of the head portion of FIG. 5.

FIG. 7 is a side view of another embodiment of a head portion of the swab of FIG. 1.

FIG. 8 is a section view taken along line 8-8 of FIG. 7.

FIG. 9 is a perspective view of the head portion of FIG. 7.

FIG. 10 is a side view of another embodiment of a head portion of the swab of FIG. 1.

FIG. 11 is a top view of the head portion of FIG. 10.

FIG. 12 is a side view of another embodiment of a head portion of the swab of FIG. 1.

FIG. 13 is a top view of the head portion of FIG. 12.

FIG. 14 is a side view of another embodiment of a head portion of the swab of FIG. 1.

FIG. 15 is a top view of the head portion of FIG. 14.

FIG. 16 is a perspective view of the head portion of FIG. 14.

FIG. 17 is a top view of another embodiment of a swab.

FIG. 18 is a side view of the swab of FIG. 17.

FIG. 19 is a top detail view of the head of the swab of FIG. 17.

FIG. 20 is a side detail view of the head of the swab of FIG. 17.

FIG. 21 is an end detail view of the head of the swab of FIG. 17.

FIG. 22 is a perspective view of a mold for the swab of FIG. 17.

FIG. 23 is a top view of another embodiment of a swab.

FIG. 24 is a side view of the swab of FIG. 23.

FIG. 25 is a detailed side view of the swab of FIG. 23.

FIG. 26 is a detailed top view of the swab of FIG. 23.

FIG. 27 is a section view taken along line 27-27 of FIG. 26.

FIG. 28 is a top view of another embodiment of a swab.

FIG. 29 is a side view of the swab of FIG. 28.

FIG. 30 is a detailed side view of the swab of FIG. 28.

FIG. 31 is a detailed top view of the swab of FIG. 28.

FIG. 32 is a perspective view of another embodiment of a swab.

FIG. 33 is a perspective view of another embodiment of a swab.

FIG. 34 is a photograph showing swabs after being used in an autoclave.

FIG. 35 is a top view of another embodiment of a swab.

FIG. 36 is a side view of the swab of FIG. 35.

FIG. 37 is a detailed top view of the swab of FIG. 35.

FIG. 38 is a detailed side view of the swab of FIG. 35.

FIG. 39 is an end view of the swab of FIG. 35.

FIGS. 40-42 are photographs of the swab of FIG. 35.

FIG. 43 is a perspective view of the swab of FIG. 35.

FIGS. 44-47 are detailed views of the head of the swab of FIG. 35.

FIG. 48 is a section view taken along line 48-48 of FIG. 46.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

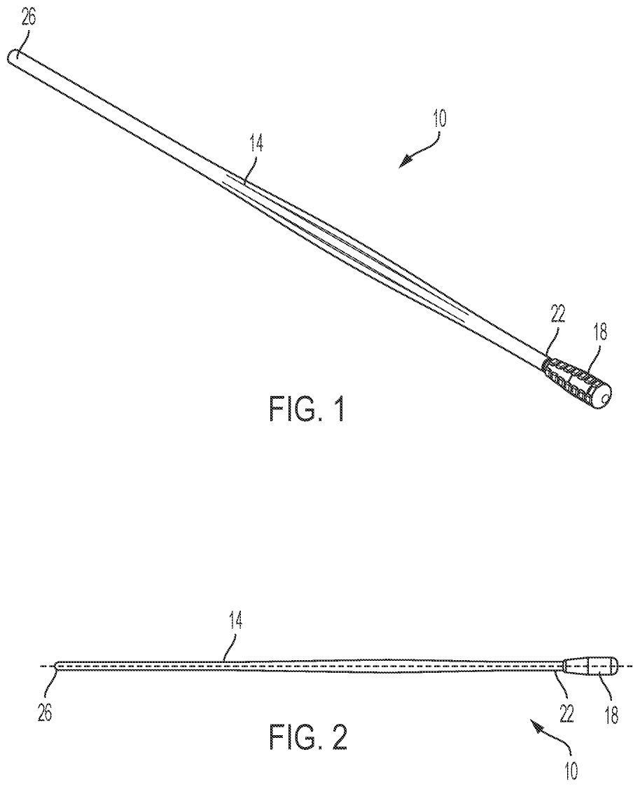

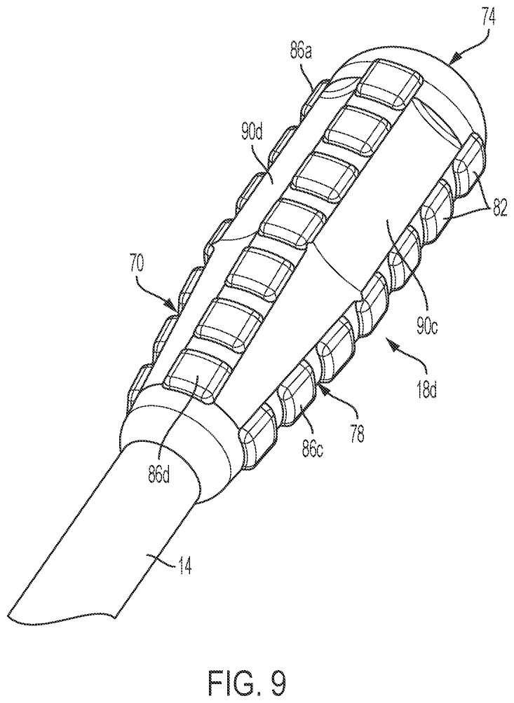

FIGS. 1-16 illustrate a swab 10 for collecting biological samples. The swab 10, in turn, includes a handle portion 14 and one or more head or tip portions 18. While the illustrated embodiment includes a single head portion 18 positioned on a first end 22 of the handle 14 (see FIGS. 1-2), it is to be understood that in alternative embodiments a second head portion 18 may be positioned on the second or opposite end 26 of the handle 14. Still further, while the illustrated handle 14 is 6 inches in length, in alternative embodiments different lengths may be used, such as, but not limited to, 8 inches, 3 inches, and the like.

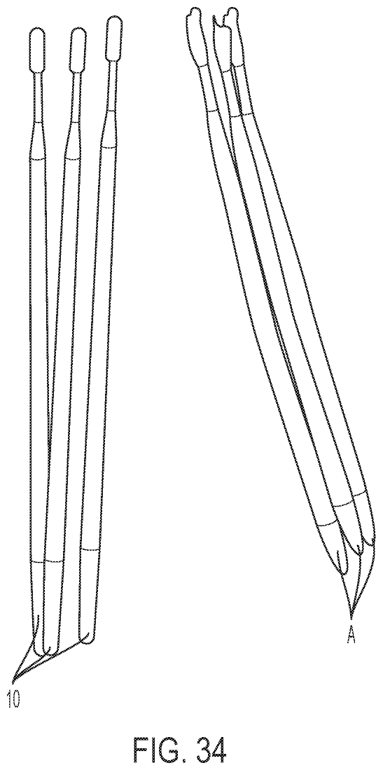

The swab 10 of the illustrated embodiment is configured to provide a relatively soft touch during the sample collecting process by spreading the interacting forces over a relatively large surface area (e.g., a relatively longer swab head 18). Furthermore, the head 18 is shaped such that it may be used for oral, pharyngeal, nasopharyngeal, and vaginal sample collection. The illustrated swab 10 is also autoclavable, being formed from medical-grade polypropylene (see FIG. 34) or other materials that can withstand the heat produced therein without excessive deformation or losing the ability to operate as intended. By being autoclavable, the swab 10 may be disinfected and reused. In alternative embodiments, the heat from the autoclave may prove damaging to swabs formed from alternative materials (see swabs A in FIG. 34).

As shown in FIGS. 1-2, the handle portion 14 and the head portion 18 are formed together as a single piece of material. More specifically, the handle portion 14 and the head portion 18 are molded together, monolithically, from a non-fibrous and non-foam type material. In the illustrated embodiment, the handle portion 14 and head portion 18 are molded together from a material such as, but not limited to, a thermoplastic elastomer (TPE), polyethylene, polypropylene, silicone, and the like. In still other embodiments, the swab 10 may be formed using a cold molding process.

In some alternative embodiments, the head portion 18 of the swab 10 may be molded onto an existing handle portion 14. In such embodiments, the head portion 18 may be formed from a non-fibrous and non-foam type material including, but not limited to, PTE, polyethylene, polypropylene, silicone and the like. Furthermore, the handle portion 14 may be formed from the same or other materials such as, but not limited to, wood, PTE, polyethylene, polypropylene, silicone, and the like.

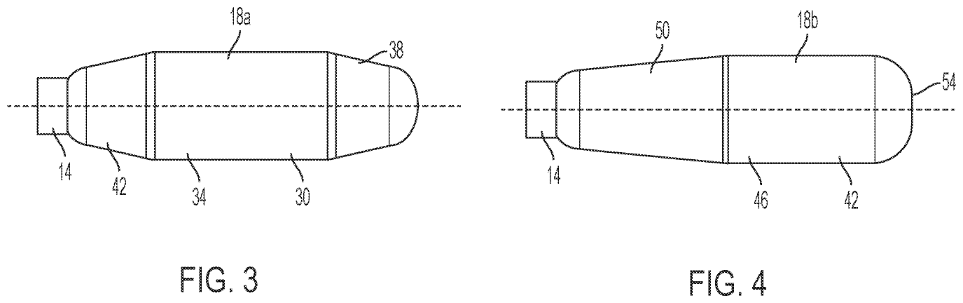

FIG. 3 illustrates a first embodiment of the head portion 18a. The head portion 18a is formed from a single piece of non-fibrous and non-foam material and includes an elongated body 30 having a substantially cylindrical center portion 34, a tapered tip portion 38 extending from the center portion 34 away from the handle portion 14, and a tapered base portion 42 extending from the center portion 34 toward the handle portion 14. As shown in FIG. 3, the tapered tip portion 38 also includes a rounded distal end. In the first embodiment of the head portion 18a, the average cross-sectional area of the head portion 18a taken normal to the axis 62 is greater than the average cross-sectional area of the corresponding handle portion 14.

FIG. 4 illustrates a second embodiment of the head portion 18b. The head portion 18b is formed from a single piece of non-fibrous and non-foam material and includes an elongated body 42 having a substantially cylindrical portion 46 and a tapered portion 50 extending from the cylindrical portion 46 toward the handle portion 14. The head portion 18b also includes a rounded tip 54.

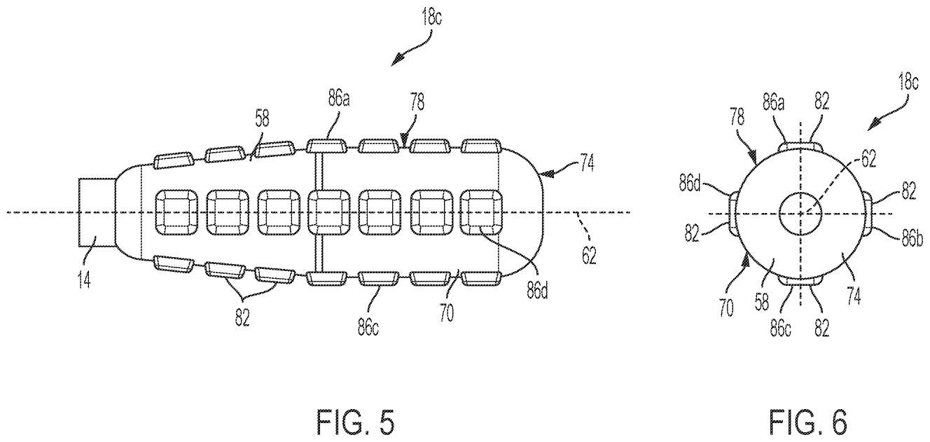

FIGS. 5-6 illustrate a third embodiment of the head portion 18c. The head portion 18c includes an elongated body 58 formed from a non-fibrous and non-foam type material having a central axis 62 extending therethrough. When formed, the axis 62 of the head portion 18c is generally co-axial with the handle portion 14.

The head portion 18c includes a body 58, and one or more protrusion 82 extending axially outwardly from the body 58. The body 58 of the head portion 18c also includes an exterior surface 70. In the illustrated embodiment the exterior surface 70 includes an end surface 74 oriented substantially normal to the axis 62 and a side surface 78 extending from the end surface 74 generally parallel to the axis 62. As shown in FIG. 5, the side surface 78 is substantially annular in shape having a substantially constant diameter over a first axial length and slightly tapering as it approaches the handle portion 14.

The protrusions 82 of the head portion 18c each extending radially outwardly from the exterior surface 70. In the illustrated construction, each protrusion 82 is substantially square in shape having flat or slightly curved exterior surfaces and rounded edges. However, in alternative embodiments, other shapes may be used. In still other embodiments, a combination of different shaped protrusions may be used on a single swab head.

As shown in FIG. 6, the protrusions 82 of the head portion 18c are generally oriented in four groups 86a, 86b, 86c, 86d, each positioned equally about the circumference of the side surface 78 (e.g., approximately 90 degrees apart from one another). Each group 86a, 86b, 86c, 86d, in turn, includes a plurality of protrusions 82 oriented in a straight line parallel to the axis 62 (e.g., along the axis length of the body 58) at even intervals.

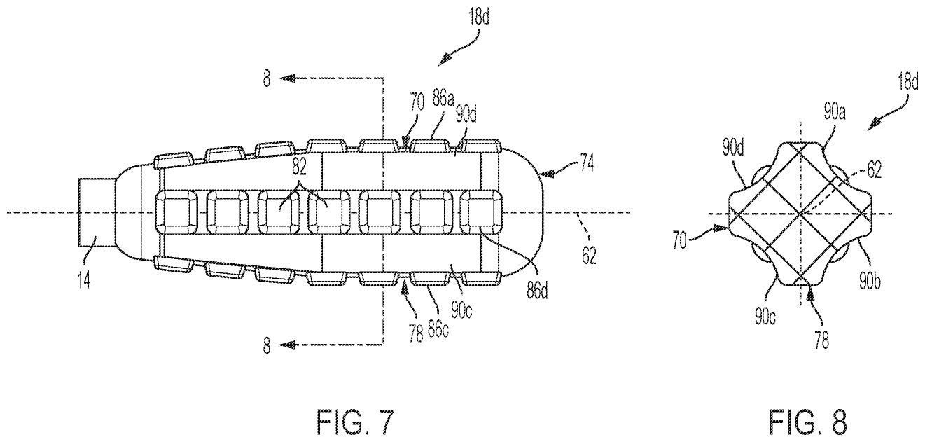

FIGS. 7-9 illustrate another embodiment of the head portion 18d. The head portion 18d is substantially similar to the head portion 18c so only the differences will be discussed herein. The body 58 of the head portion 18d includes a plurality of elongated grooves 90a, 90b, 90c, 90d formed into the exterior surface 70. More specifically, each groove 90a, 90b, 90c, 90d is generally positioned 90 degrees from one another and extends axially along the length of the body 58 (e.g., parallel to the axis 62). Each groove 90a, 90b, 90c, 90d, in turn, forms a concave surface having a substantially constant radius along its entire axial length.

In the illustrated embodiment, each group of protrusions 86a, 86b, 86c, 86d is positioned between two grooves 90a, 90b, 90c, 90d producing an alternating “groove, group, groove, group” pattern along the circumference of the exterior surface 70 (see FIG. 8). However, in alternative embodiments this may be changed and more or fewer groups and/or grooves may be used. In still other embodiments, different sizes and shapes of grooves may be present such as, but not limited to, dimples, circumferentially extending grooves, and the like. In still other embodiments, the grooves may only extend along a portion of the axial length of the body 58.

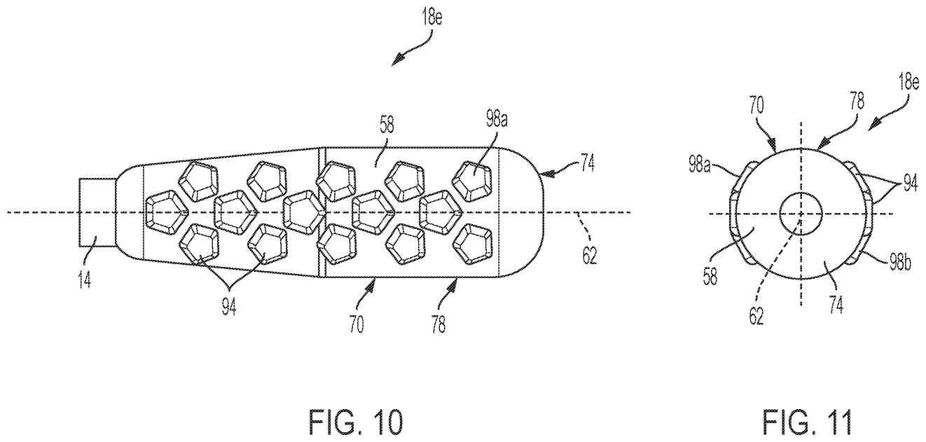

FIGS. 10-11 illustrate another embodiment of the head portion 18e. The head portion 18e is substantially similar to the head portion 18c so only the differences will be discussed herein. The protrusions 94 of the head portion 18e are substantially pentagonal in shape having flat or slightly curved exterior surfaces and some rounded edges. While the protrusions 94 of the head portion 18e are pentagonal, in alternative embodiments, different polygonal shapes may also be present such as, but not limited to, hexagons, trapezoids, and the like.

The protrusions 94 of the head portion 18e are generally positioned in two groups 98a, 98b, each positioned equally about the circumference of the outer surface 70 (e.g., approximately 180 degrees apart). Each group 98a, 98b, in turn, includes a plurality of protrusions 94 oriented in an array extending parallel to the axis 62 (e.g., along the axial length of the body 58). Specifically, the protrusions 94 are positioned in an alternating 2:1:2:1 pattern so that the protrusions 94 of a given row are positioned between the protrusions 94 of adjacent rows (e.g., protrusions 94 are aligned with the gaps between the protrusions 94 of the adjacent rows).

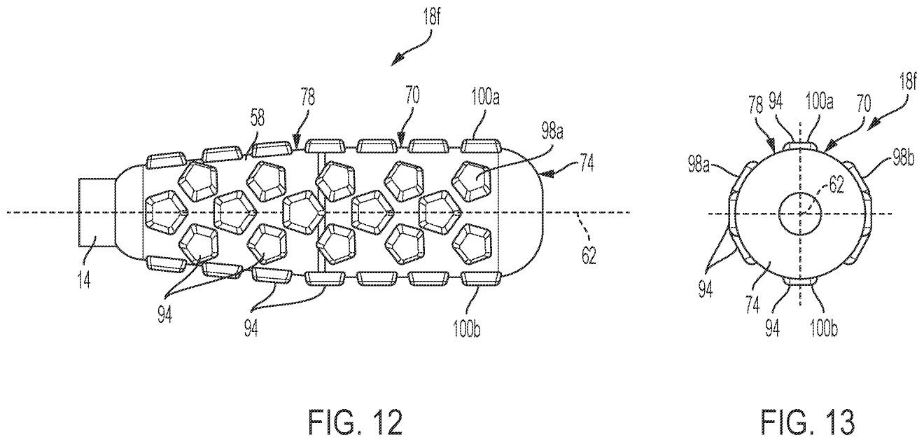

FIGS. 12-13 illustrate another embodiment of the head portion 18f Head portion 18f is substantially similar to head portion 18e so only the differences will be discussed herein. Head portion 18f includes a pair of secondary protrusion groups 100a, 100b positioned opposite one another (e.g., 180 degrees apart) and positioned between groups 98a, 98b (e.g., 90 degrees apart from groups 98a, 98b). Each secondary group 100a, 100b includes a plurality of protrusions 94 oriented in a straight line along the axial length of the head 18f (e.g., parallel the axis 62). While the illustrated protrusions 94 are similar to the protrusions 94 of groups 98a, 98b; in alternative embodiments the size and shape of the protrusions 94 may differ between groups 98a, 98b and secondary groups 100a, 100b.

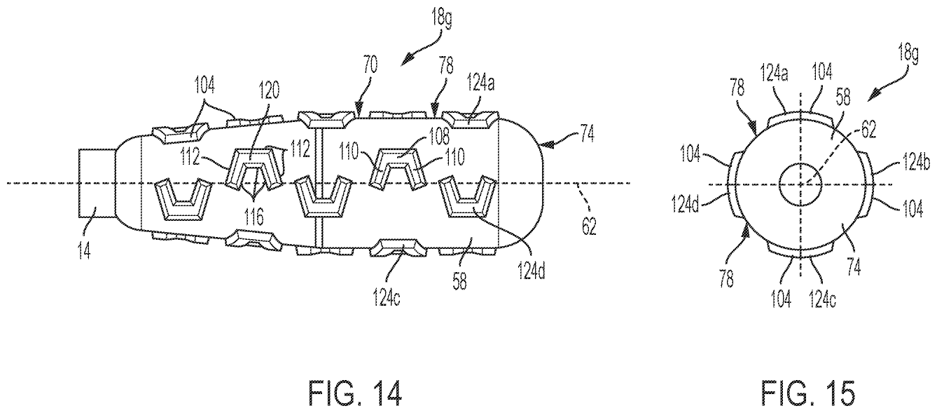

FIGS. 14-16 illustrate another embodiment of the head portion 18g. Head portion 18g is substantially similar to head portion 18c so only the differences will be discussed herein. The head portion 18g includes a plurality of protrusions 104. The protrusions 104 are generally “U” shaped having a base wall 108 and two end walls 110 extending from the base wall 108. All three walls 108, 110, in turn, protrude radially outwardly from the outer surface 70 of the body 58.

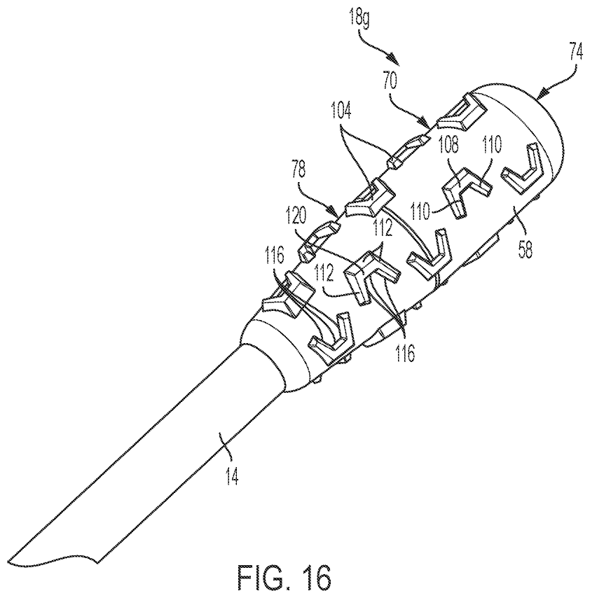

As shown in FIGS. 14 and 16, each wall 108, 110 of the protrusions 104 have exterior edges 112, interior edges 116, and a generally flat top surface 120. As shown in FIG. 14, the interior edge 116, together, form a concave region at least partially enclosed by the walls 108 of the protrusions 104. As shown in FIG. 16, the exterior edges 112 form a first edge angle relative to a radial datum of the axis 62. In contrast, the interior edges 116 form a second edge angle that is less than the first edge angle. By doing so, the interior of the protrusions 104 present a more aggressive surface (e.g., more perpendicular to the exterior surface 70) so that, during the swabbing action, biological material is scraped clear from the surface being swabbed and is collected within the concave center of the “U” shape. In contrast, the exterior edges 112 provide a greater edge angle so that the exterior edges 112 more easily slide across the surface being swabbed.

As shown in FIG. 14, the protrusions 104 of the head portion 18g are generally oriented in four groups 124a, 124b, 124c, 124d, each positioned equally about the circumference of the exterior surface 70 (e.g., approximately 90 degrees apart from one another). Each group 124a, 124b, 124c, 124d, in turn includes a plurality of protrusions 104 each oriented parallel to the axis 62 (e.g., along the axial length of the body 58) at even intervals. FIG. 14 also illustrates that the protrusions are also oriented so that the concave “open” end of the “U” shape alternates.

While the illustrated swab 10 is shown, it is understood that the protrusions and constructions styles described above may also be applied to alternative swab types and different head sizes and shapes. Furthermore, the protrusion styles and layouts may be combined or separated as needed to produce the desired biological sample collection capabilities.

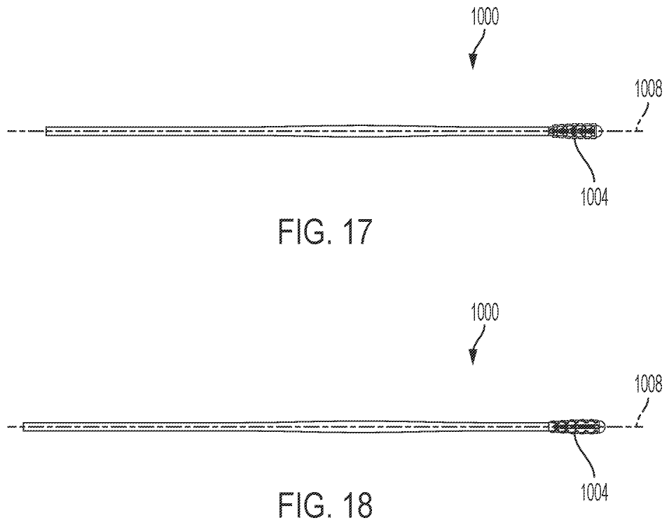

FIGS. 17-20 illustrate another embodiment of the swab 1000. The swab 1000 is substantially similar to the swab 10 described above. As such, only the differences will be discussed in detail herein. The head portion 1004 of the swab 1000 is substantially elongated in shape defining an axis 1008 therethrough. The head portion 1004 includes a body 1012 and a plurality of protrusions 1014, 1036 extending outwardly therefrom. In the illustrated embodiment, the body 1012 includes a hemispherical head portion 1016 at the distal end thereof, a cylindrical portion 1020 extending axially from the hemispherical head portion 106, and a tapered or frusto-conical portion 1024 extending radially inwardly as it extends axially from the cylindrical portion 1020 opposite the hemispherical head portion 1016. In the illustrated swab 1000, the handle 14 extends axially from the frusto-conical portion 1024 opposite the cylindrical portion 1020.

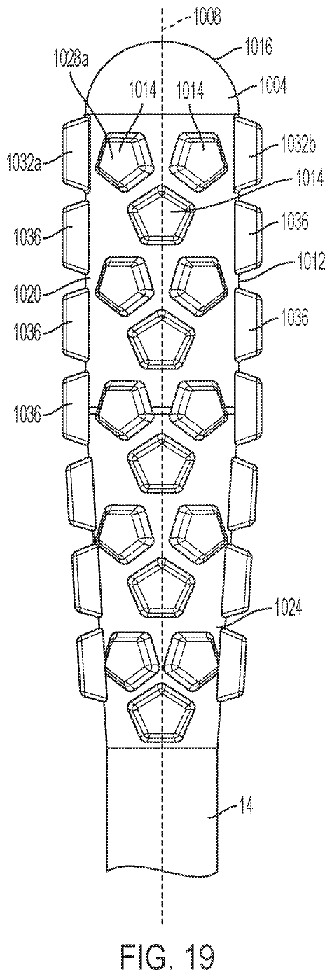

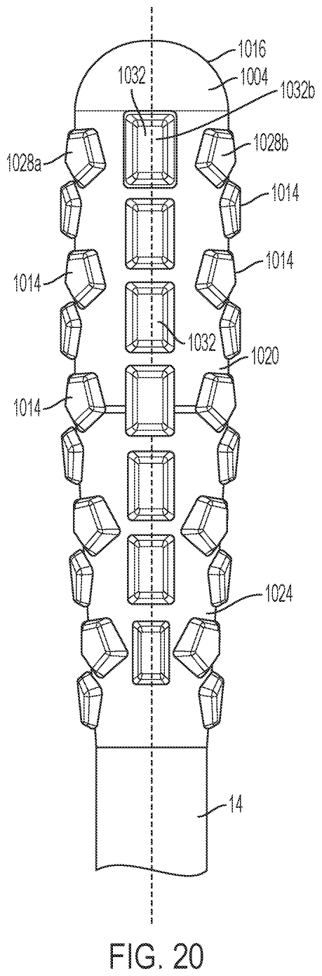

The head portion 1004 also includes a pair of primary protrusion groups 1028a, 1028b. Each primary protrusion group 1028a, 1028b includes a plurality of primary protrusions 1014 extending outwardly from the exterior surface of the body 1012 and are positioned opposite each other (e.g., 180 degrees apart). Each protrusion 1014, in turn, is substantially polygonal in shape (e.g., pentagonal) and narrows as it extends away from the body 1012. As shown in FIG. 19, the protrusions 1014 are oriented in an alternating pattern extending along the axis 1008 whereby each protrusion 1014 in a given row are located between protrusions 1014 in adjacent rows.

The head portion 1004 also includes a pair of secondary protrusion groups 1032a, 1032b. Each secondary protrusion group 1032a, 1032b includes a plurality of secondary protrusions 1036 extending outwardly from the exterior surface of the body 1012 and are positioned opposite each other (e.g., 180 degrees apart) and orthogonal to the primary protrusion groups 1028a, 1028b (e.g., 90 degrees apart). Each secondary protrusion 1036, in turn, is substantially rectangular in shape and narrows as it extends away from the body 1012. As shown in FIG. 20, the secondary protrusions 1036 are oriented in substantially axial alignment. As shown in FIGS. 19 and 20, the secondary protrusion 1036 extend further from the body 1012 than the primary protrusions 1014.

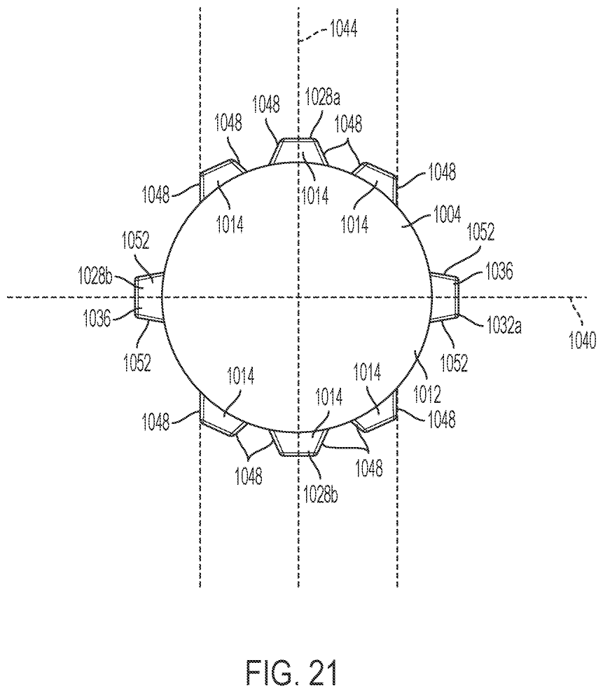

As shown in FIG. 21, the head portion 1004 defines a parting plane 1040 aligned with the axis 1008 and passing through the secondary protrusion groups 1032a, 1032b. The head portion 1004 also defines a second plane 1044 aligned with the axis 1008 and oriented perpendicular to the parting plane 1040 (see FIG. 21). As shown in FIG. 21, the sidewalls 1048 of the primary protrusions 1014 are either facing away from the parting plane 1040 or are perpendicular to the parting plane 1040. For the purposes of this application, a sidewall 1048 is facing away from the parting plane 1040 if a ray originating on the respective sidewall 1048 and extending normal therefrom does not cross the parting plane 1040. In the illustrated embodiment, the sidewalls 1052 of the secondary protrusions 1036 also face away from the parting plane 1040. Stated differently, the sidewalls 1048, 1052 of each protrusion 1014, 1036 face away from the parting plane 1040. By doing so, the protrusions 1014, 1036 are oriented and shaped such that the head portion 1004 may be molded as a single piece in a two-piece mold (e.g., the geometry of the head portion 1004 is two-piece mold compatible). Indeed, in the illustrated embodiment of FIGS. 17-21, both the handle portion and the head portion 1004 are mold compatible such that the entire swab is two-piece mold compatible.

In some embodiments, the exterior surface of the swab head 1004 may include a texture included thereon to increase the ability of the head 1004 to collect and help retain the biological sample material thereon. In such embodiments, the texture may be included on the body 1012, the primary protrusions 1014, the secondary protrusions 1036, or any combination or portion thereof. Furthermore, the texture may be imparted onto the head 1004 either during the molding process or as a separate step after the swab 1000 has been formed. In instances where a texture is applied to a portion of the head 1004 (e.g., the protrusions 1014), the exterior surface of the corresponding swab head 1004 includes a first portion having a first texture with a first roughness and a second portion having a second texture with a second roughness greater than the first roughness.

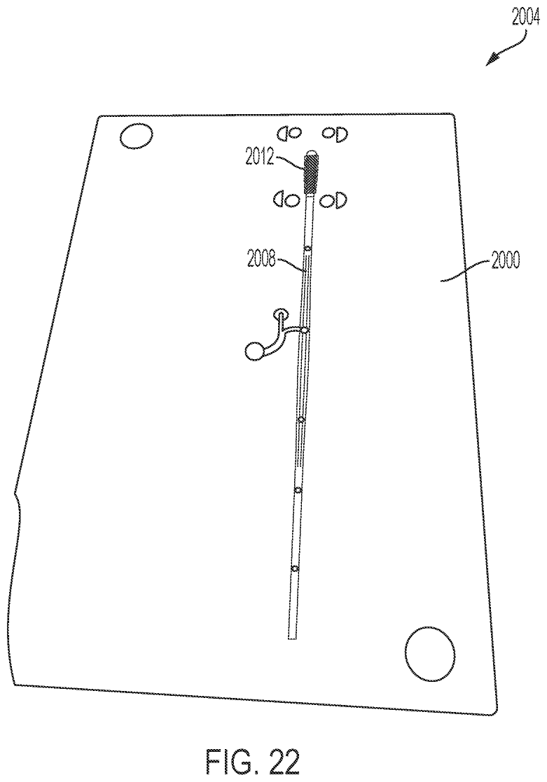

FIG. 22 illustrates one half or plate 2000 of a mold 2004 for molding the swab 1000 by an injection molding process. During manufacturing, a second half or plate 2000 of the mold 2004, similar to the first half 2000, are brought together to form a cavity 2008 therebetween with a shape generally corresponding to the shape of the swab to be formed. As shown in FIG. 22, the illustrated embodiment includes an insert 2012 such that a portion of the swab shape (e.g., the head portion and/or the handle portion) can be changed out or replaced easily and quickly. Still further, the mold 2004 may include an insert (not shown) to allow the user to select whether a second head portion is molded integrally with the handle portion opposite the first head portion. While the illustrated mold 2004 is shown having inserts 2012 to produce the head portion 1004 and handle portion of the swab 1000, it is understood that other mold compatible swab designs illustrated herein may also be manufactured in the same or a similar manner.

In some embodiments, the swab 1000 may be molded in a single or multi-step process. For example, in the illustrated embodiment, the head portion 1004 and the handle portion 14 of the swab 1000 are molded together during a single-step molding process in a single mold cavity (discussed above) to produce a monolithic swab. However, in alternative embodiments, the body 1012 of the head portion 1004 and the handle portion 14 may be molded together during a single-step molding process in a single mold cavity to produce a monolithic structure. Subsequently, the protrusions 1014, 1036 may be overmolded onto the monolithic structure to produce the finished swab. In such embodiments, the protrusions 1014, 1036 may be overmolded using a second material different than the first material, or using the same material as the underlying structure. In still other embodiments, the entire swab may be molded as a single monolithic structure in a single step using medical grade polypropylene.

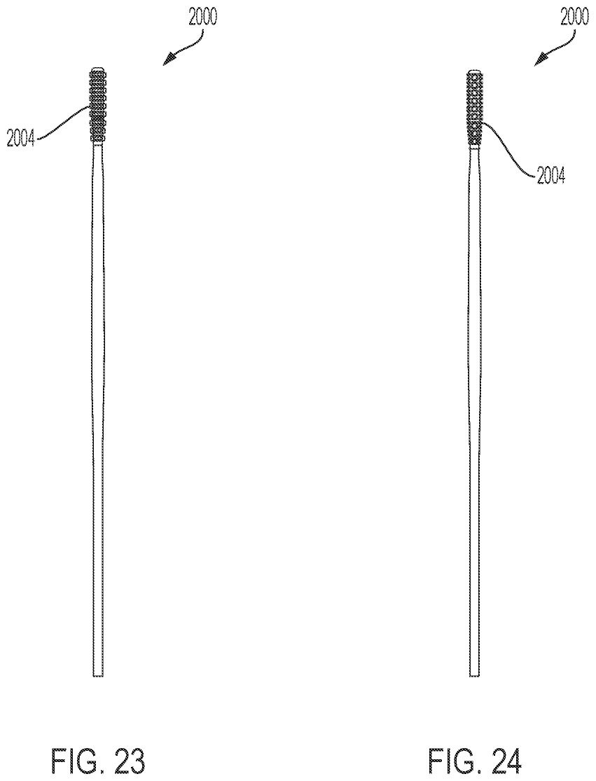

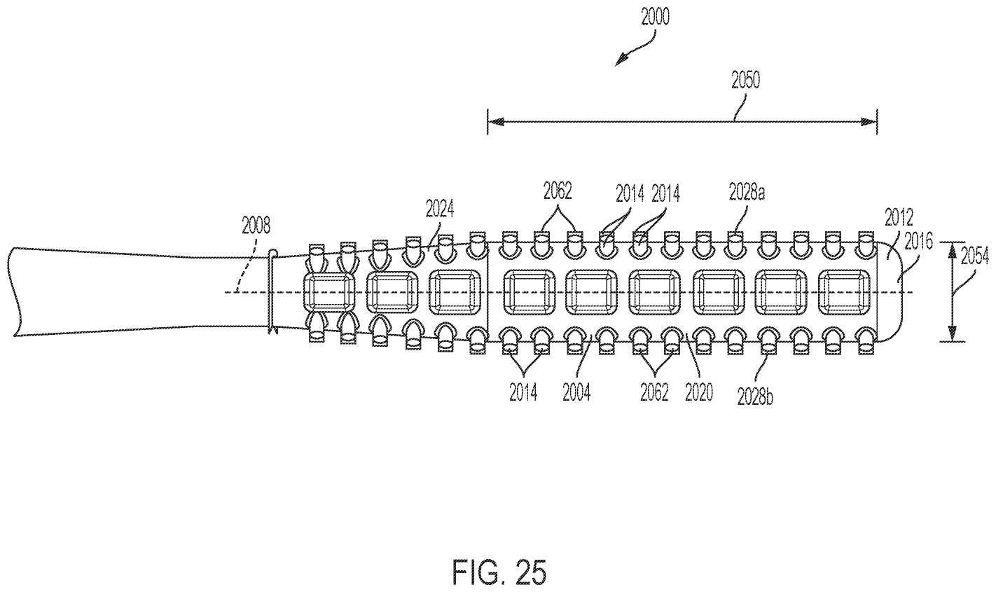

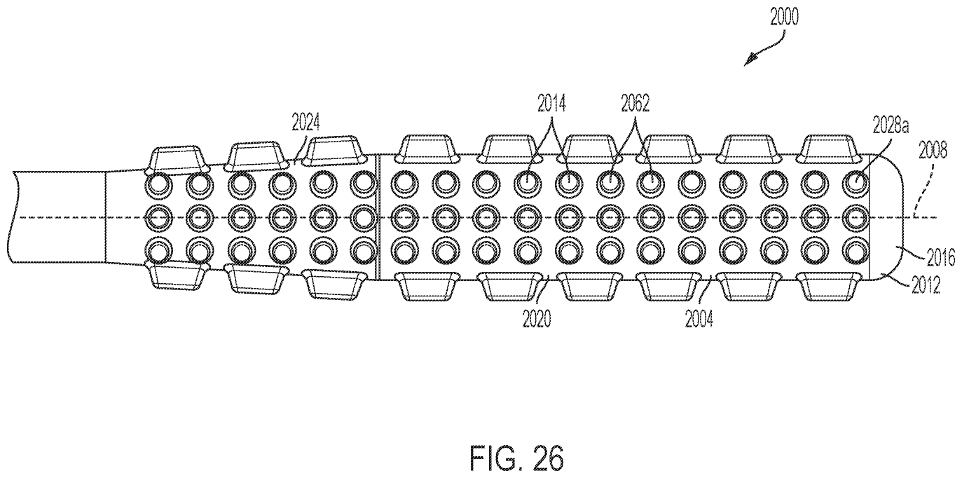

FIGS. 23-27 illustrate another embodiment of the swab 2000. The swab 2000 is substantially similar to the swab 1000 of FIG. 17-20 described above. As such, only the differences will be discussed in detail herein. The head portion 2004 of the swab 2000 is substantially elongated in shape defining an axis 2008 therethrough. The head portion 2004 includes a body 2012 and a plurality of primary protrusions 2014 and a plurality of secondary protrusions 2036 extending outwardly therefrom. In the illustrated embodiment, the body 2012 includes a rounded head portion 2016 at the distal end thereof, a cylindrical portion 2020 extending axially from the rounded head portion 2016, and a tapered or frusto-conical portion 2024 extending radially inwardly as it extends axially from the cylindrical portion 2020 opposite the rounded head portion 2016. In the illustrated embodiment, the axial length 2050 of the cylindrical portion 2020 is at least three times the diameter 2054 of the cylindrical portion 2020.

The head portion 2004 also includes a pair of primary protrusion groups 2028a, 2028b. Each primary protrusion group 2028a, 2028b includes a plurality of primary protrusions 2014 extending outwardly from the exterior surface of the body 2012 and are positioned opposite each other (e.g., 180 degrees apart). Each protrusion 2014, in turn, is substantially cylindrical in shape having a flat or near-flat end 2062.

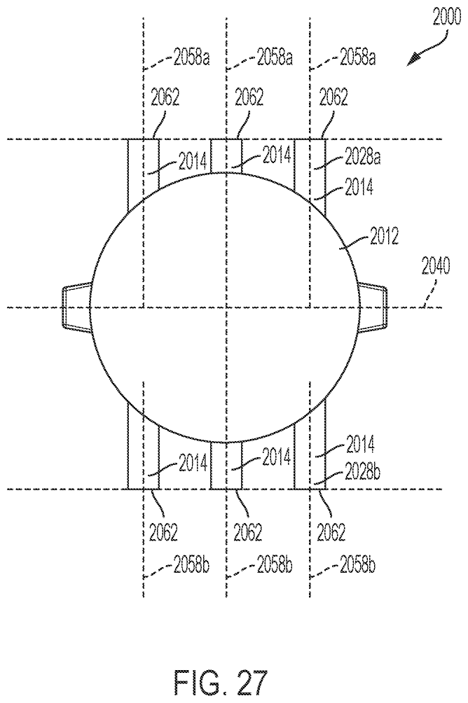

As shown in FIG. 27, each protrusion 2014 defines an axis 2058a, 2058b extending therethrough. Each axis 2058a, 2058b, in turn, is substantially parallel with the axis 2058a, 2058b of all other protrusions 2014 in that particular group 2028a, 2028b (e.g., all protrusions 2014 of the first group 2028a are parallel with each other and all protrusions 2014 of the second group 2028b are parallel with each other). In the illustrated embodiment, the axis 2058a, 2058b of each protrusion 2014 is substantially normal to the parting plane 2040. Also shown in FIG. 27, the illustrated protrusions 2014 are shaped so that the ends 2062 of each protrusion 2014 all terminate along a similar plane. More specifically, the similar plane includes a plane that is oriented parallel to the parting plane 2040. In alternative embodiments, each protrusion 2014 may terminate along a different plane (e.g., a different distance from the parting plane 2040). In still other embodiments, each row of protrusions 2014 may have similar heights.

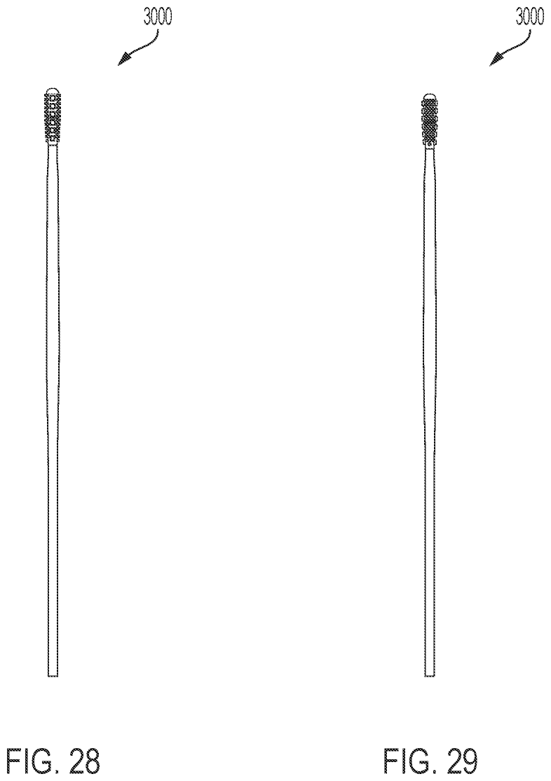

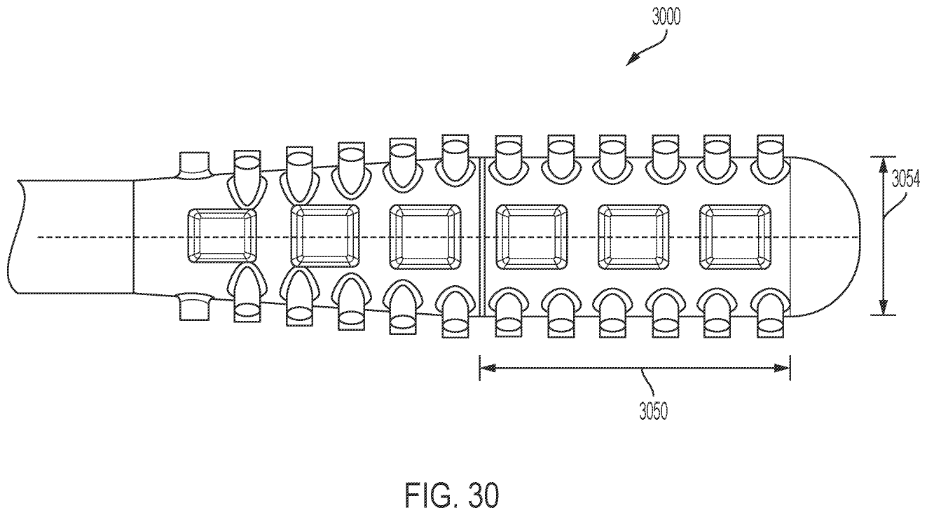

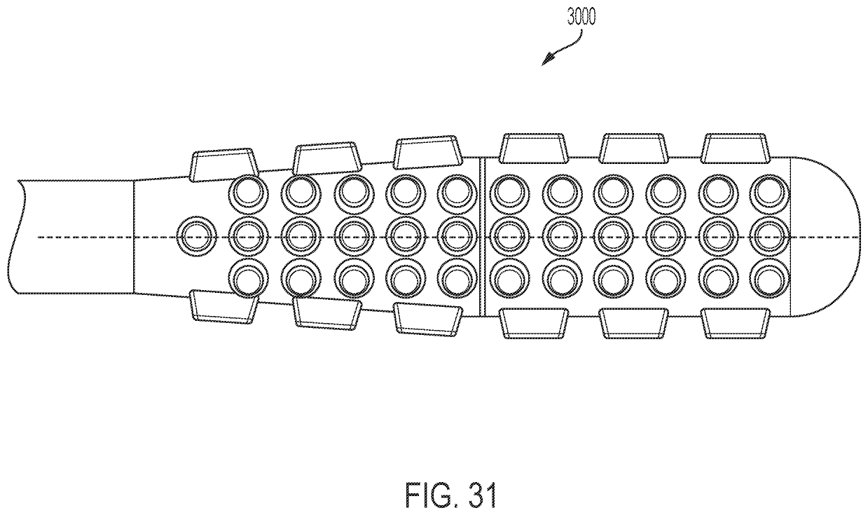

FIGS. 28-31 illustrate another embodiment of the swab 3000. The swab 3000 is substantially similar to the swab 2000 described above. The cylindrical portion 3020 of the swab 3000 has a shorter axial length 3050 relative to the diameter 3054.

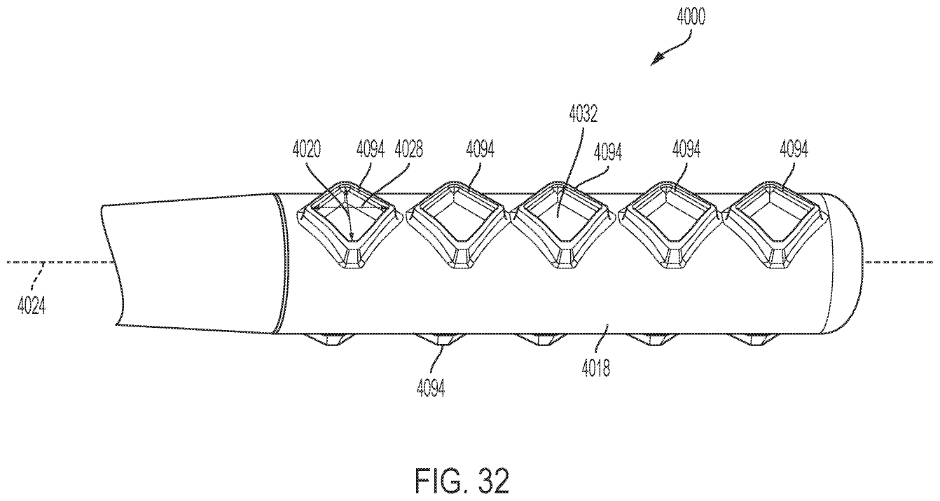

FIG. 32 illustrates another embodiment of the swab 4000. The swab 4000 includes a head 4018 substantially similar to the swab head 18e described above. The protrusions 4094 are substantially diamond shaped having a greater axis 4020 oriented substantially perpendicular to the axis 4024 and a minor axis 4028 oriented substantially parallel to the axis 4024. Each diamond shape is substantially hollow having an exterior wall providing the diamond shape with a depressed or recessed interior 4032. While the illustrated protrusions 4094 are wider perpendicular to the axis 4024, it is understood that in alternative embodiments the protrusions 4094 may be oriented so that the greater axis 4020 is parallel to the axis 4024.

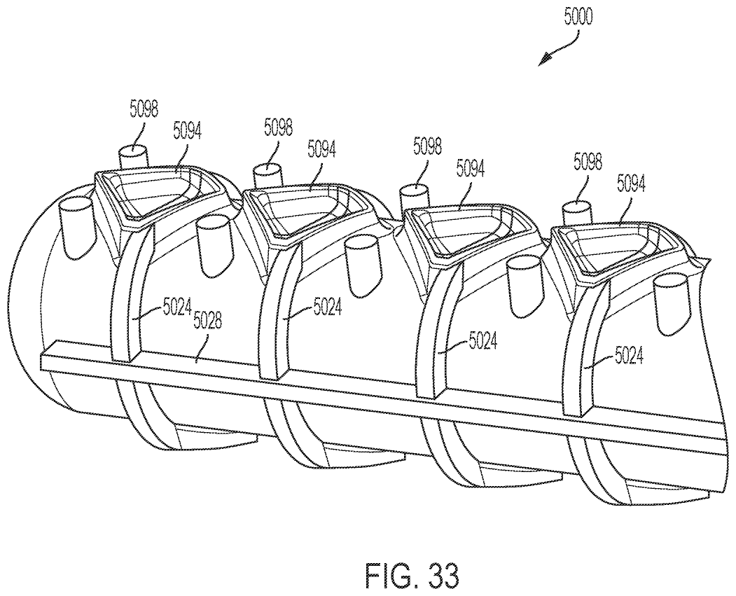

FIG. 33 illustrates another embodiment of the swab 5000. The swab 5000 is substantially similar to the swab 4000 described above. The swab 5000 includes a first set of protrusions 5094 that are substantially diamond shaped and positioned 180 degrees apart. The swab 5000 also includes a set of substantially cylindrical protrusions 5098 extending from the head 5018 substantially parallel to the first set of protrusions 5094. As shown in FIG. 33, the first and second sets of protrusions 5094, 5098 substantially extend to the same height.

The swab 5000 also includes a set of annular ribs 5024 extending circumferentially around the head 5018. As shown in FIG. 33 the ribs 5024 are substantially aligned with the first set of protrusions 5094.

The swab 5000 also includes a set of axial ribs 5028 extending axially along the body of the head 5018. As shown in FIG. 33, the ribs 5028 are oriented substantially 90 degrees from the first set of protrusions 5094.

To use the swab for nasopharyngeal swabbing, the user inserts the head of the swab into the nasal cavity, rotates the swab approximately 360 degrees, then removes the head of the swab from the nasal cavity. The size and shape of the head of the swab is configured such that the rotation of the head within the nasal cavity causes the biological material to become attached thereto. The biological material may then be removed from the swab and placed into a container (e.g., a test tube) for further testing.

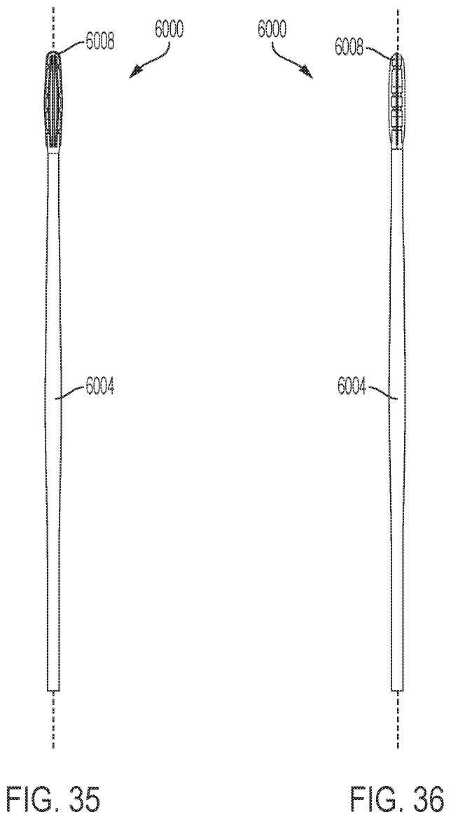

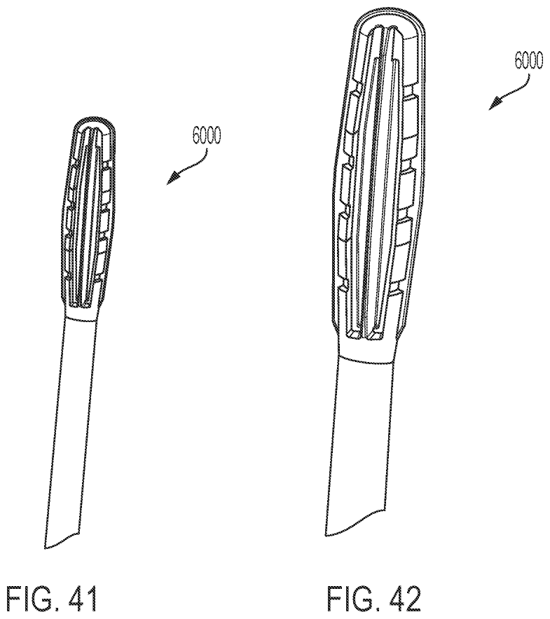

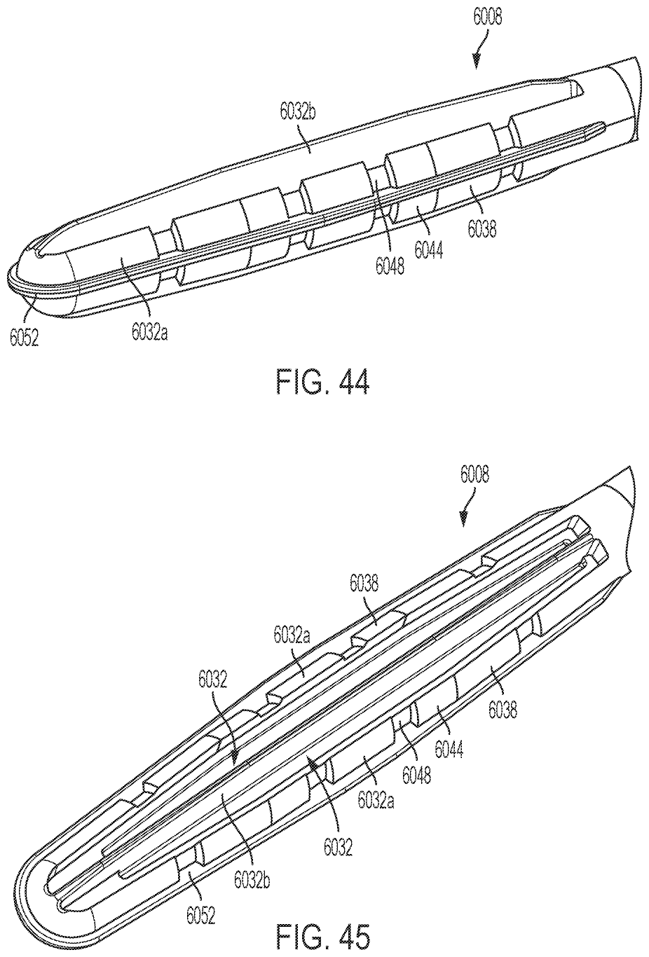

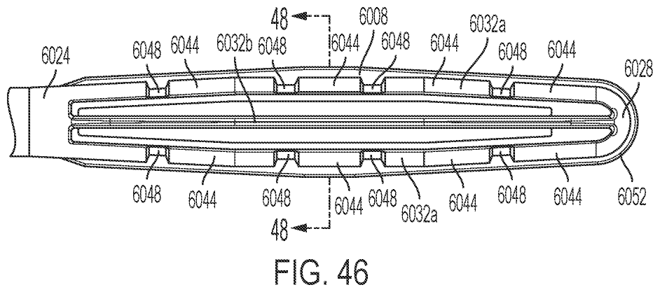

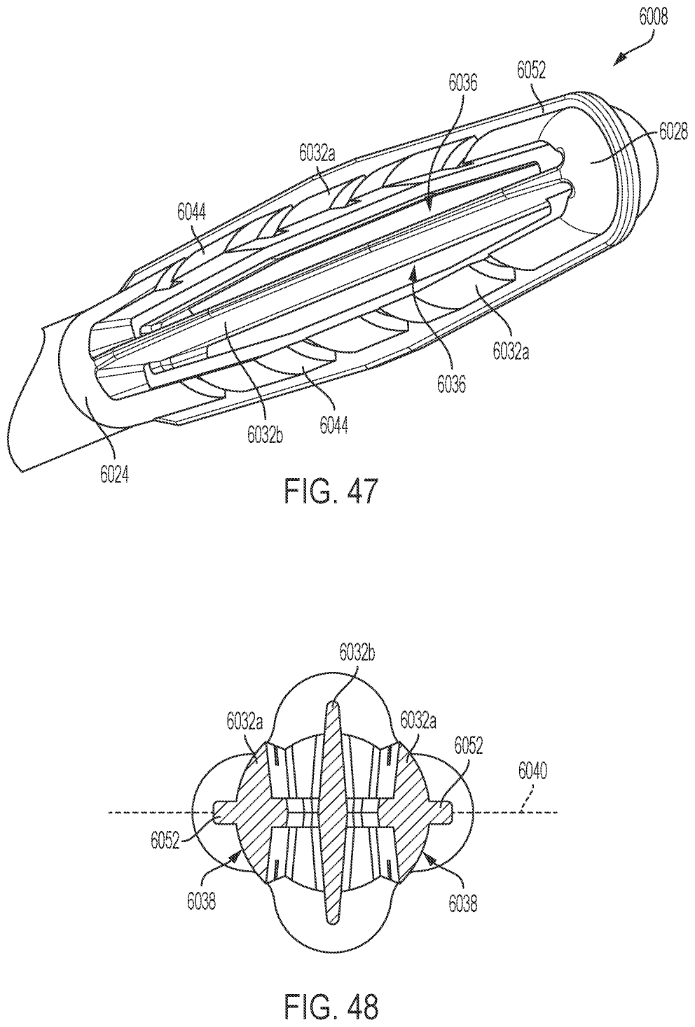

FIGS. 35-48 illustrate another embodiment of a swab 6000. The swab 6000 is configured to grab more cells from the sample material and retain the cells on the head until released in a biological sample holder (e.g., a test tube). The swab 6000 is substantially similar to the swabs discussed above so only the differences will be discussed herein. To help tackle this issue, the swab at least partially defines at least one internal pocket that can be easily filled with a volume of oral fluid and retain the oral fluid within the pocket by surface tension during the cheek scrubbing process or nasal scrubbing process. The retained liquid, in turn, acts like a magnet that pulls cells in and keeps the cells stuck and piling up during the collection process adding more captured cells. After the sample has been collected, the liquid within the pocket may be disrupted when it goes into the biological sample holder by the squeezing/scrubbing functionality of the holder causing the retained cells and material to be released. Furthermore, the head design of the swab 6000 allows the exterior surface of the head 6008 to flex inwards when it enters the nasal cavity, thereby making it less invasive and easier to insert and capture more cells on the way out. Therefore, the illustrated swab achieves greater cell capture and comfort than alternative designs.

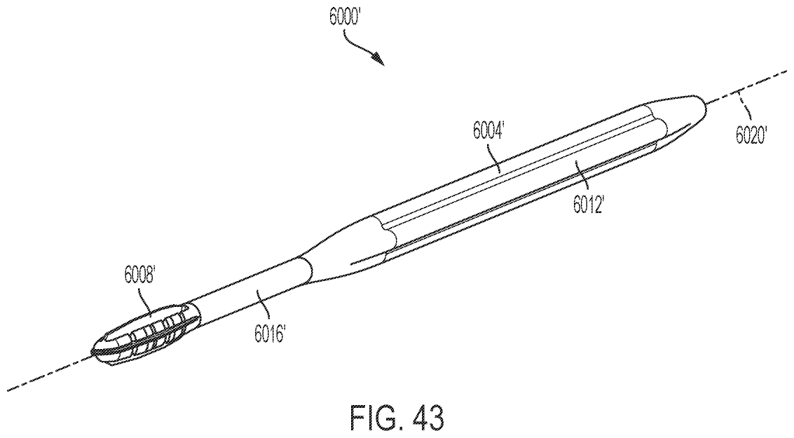

As shown in FIG. 43, the swab 6000 includes a handle portion 6004, a head portion 6008, and defines an axis 6020 extending along the length thereof. In the illustrated embodiment, the handle portion 6004 is an elongated shape.

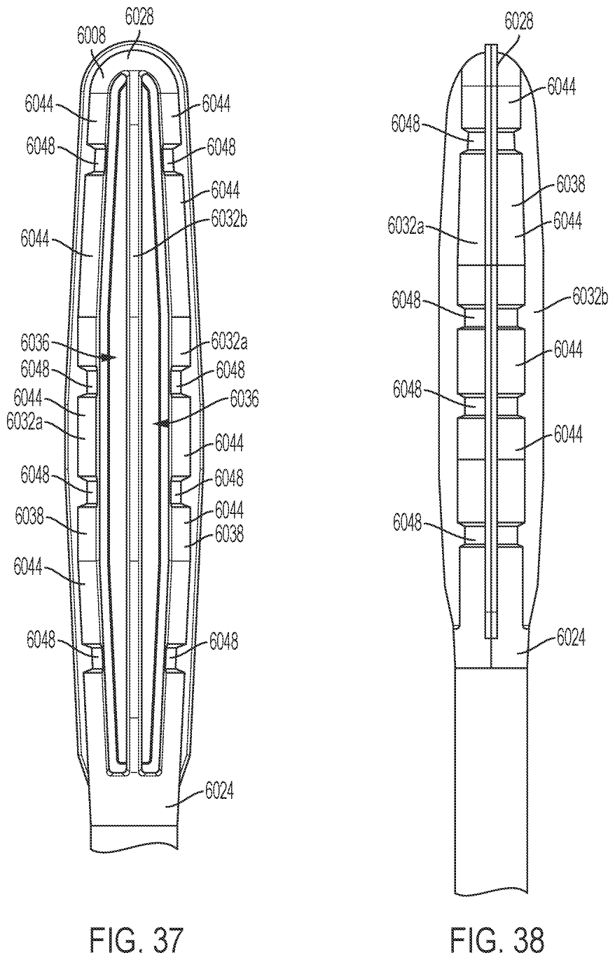

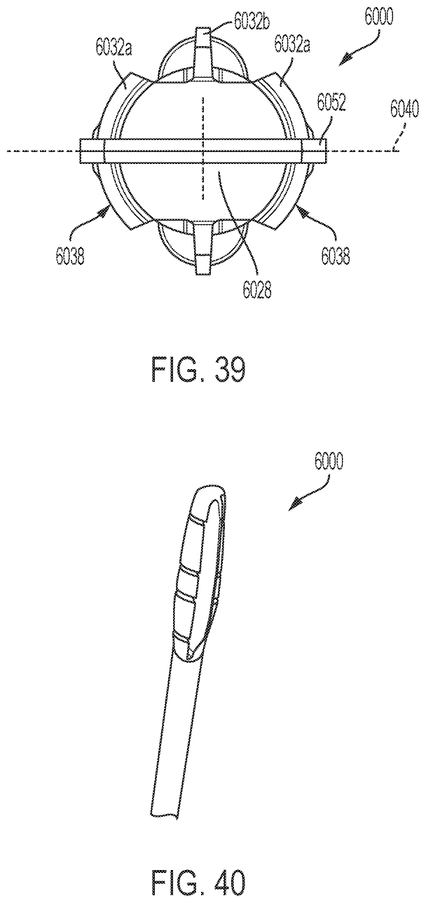

The head portion 6008 of the swab 6000 includes a base 6024, a tip 6028, and a plurality of legs 6032a, b extending between the base 6024 and the tip 6028. As shown in FIG. 37, the legs 6032a. b are separated from one another over at least a portion of their length to from gaps 6036 therebetween (e.g., a fluid pocket). In the illustrated embodiment, the legs 6032a, b are joined near the base 6024 (e.g., at the nexus with the handle portion 6004) and the tip 6028 such that the gaps 6036 extend nearly the entire axial length of the head portion 6008. The legs 6032a, b are also oriented so that they are spread out over a first plane 6040 (see FIG. 39) such that each gap 6036 is open both above and below (e.g., perpendicular to the first plane 6040. Although not shown, in alternative embodiments one or more cross-braces may be present.

In the illustrated embodiment, the head portion 6008 includes a pair of exterior legs 6032a and at least one interior leg 6032b positioned between the pair of exterior legs 6324a. As shown in FIG. 37, the exterior legs 6032a each have an outwardly facing surface 6038 that includes one or more pads 6044 separated by gullets 6048. In the illustrated embodiment, the exterior surface 6036 is substantially arcuate in shape, however in alternative embodiments other sizes and shapes may be present. During use, the gaps 6036 act as fluid pockets acting to retain oral fluid therein whereby cells are drawn into and retained within the fluid contained in the gaps 6036. To remove the cells, the exterior legs 6032a, c, may be “squeezed” toward the interior leg 6032b.

The one or more interior legs 6032b are substantially planar being oriented substantially perpendicular to the first plane 6040 (see FIG. 39). The interior leg 6032b is also sized such that it extends, in a direction generally perpendicular to the exterior legs 6032b, beyond the exterior legs 6032a in both directions.

The head portion 6008 of the swab 6008 also includes a rib 6052 extending along the periphery of the head 6008. The rib 6052 is oriented substantially parallel to the first plane 6040 and extends outwardly beyond the exterior surface 6036 of the exterior legs 6032a. As shown in FIG. 48, the rib 6052 begins near the base 6024 proximate one exterior leg 6032a, extends along the leg 6032a to the tip 6028, wraps around the tip 6028, and extends along the opposite exterior leg 6032a where it terminates proximate the base 6024. As shown in FIG. 48, the rib 6052 may also extend radially inwardly from the exterior legs 6032a such that it is at least partially positioned within the gaps 6036.

In the illustrated embodiment, the head portion 6008 also includes at least two different textures on the exposed surfaces thereof. More specifically, the head portion includes a first portion of the exposed surfaces having a first, lower-friction texture level and a second portion of the exposed surfaces having a second, higher-friction texture level that is different from the first texture level. In the illustrated embodiment, the head 6008 is configured such that each of the pads 6044 and tip 6028 have the second texture level while the rest of the exposed surfaces of the head portion 6008 include the first texture level. In some embodiments, the entire swab 6000 may be molded so that all of the exterior surfaces have the first texture level with the second texture level being added after the molding process is complete. In other embodiments, the first and second texture levels may be applied as part of the molding process (e.g., via the mold itself). In the illustrated embodiment, the second texture level is approximately 32 RMS (Root Mean Square roughness).

To use the swab 6000, the user grasps the handle portion 6004 and inserts the head portion 6008 into the desired cavity (e.g., the nasal cavity, and the like). The user may then rotate or otherwise maneuver the head portion 6008 within the cavity to collect the desired biological sample. When the head portion 6008 is maneuvered, a volume of liquid (e.g., mucus, oral fluids, and the like) are collected within the two pockets or gaps 6036 and retained therein at least partially by the surface tension of the liquid. A combination of the geometry of the exterior surface and the retained liquid then dislodges and retains cells within the liquid and on the surface of the swab head portion 6008.

With the sample collected, the user may then remove the head portion 6008 from the passage. With the head removed, the use may then release the sample into a biological sample container (e.g., a test tube, vial and the like). To do so, the geometry of the container may be used or the exterior legs 3062a of the head portion 6008 may be “squeezed” inwardly to force any fluid contained within the gaps 6036—and the cells retained within the fluid—to be ejected. Any cells retained on the exterior surface of the swab head 6008 may also be released at this time using similar methods. In instances where the swab 6000 is to be reused, the swab may be placed in a autoclave and cleaned.

Furthermore, the exterior legs 3062a of the head portion 9008 are configured to “flex” during use. Specifically, the legs 3062a can compress inwardly toward the interior leg 3062b, decreasing the overall width of the head portion 2008. This allows the head portion 9008 to more easily accommodate the contours and sizes of the surfaces against which it is pressed to gather a biological sample.

To manufacture the swab 6000, the user may first take two mold halves (discussed above) and assemble them to at least partially enclose a mold cavity therein (see FIG. 22). The mold cavity, in turn, defines both the swab head portion 6008 and the swab handle portion 6004. In the illustrated embodiment, the part line between the two mold halves are coincident with the first plane 6040 of the swab 6000. With the cavity enclosed, a first material (e.g. a thermosplastic elastomer, polyethylene, polypropylene, silicone, and the like) is injected into the cavity to form a monolithic part including both the swab head 6008 and swab handle 6004. The resulting part may then be ejected from the cavity.

The head portion 6008 and handle portion 6004 of the swab 6000 are both sized and shaped so that they are two-piece mold compatible along the first plane 6040 (e.g., first plane 6040 as a parting plane). As such, the head portion 6008 and handle portion 6004 may both be molded together as a single piece of monolithic material during a single-stage casting process (as discussed above). While not shown, one or more portions of the head portion 9008 and/or handle portion 6004 may be overmolded onto a sub-structure as well. Such overmolded portions may be formed from a second material different than the first material.

FIG. 43 illustrates an alternative embodiment of the handle portion 6004′ of the swab 6000′. The handle portion 6004′ includes a grip portion 6012′ forming an ergonomic grip, and a neck portion 6016′ extending between the grip portion 6012′ and the head portion 6008′. As shown in FIG. 43, the grip portion 6012′ of the handle portion 6004 includes a plurality of rounded contours oriented parallel to the axis 6020′ of the swab 6000′ (e.g., three triangularly oriented cylindrical elements). However, in alternative embodiments different ergonomic shapes may be used.

In some embodiments, the neck portion 6016′ of the handle 6004′ is configured to flex or provide flexibility to the swab 6000′ so that the head portion 6008′ can travel along passages (e.g., naval passages and the like) and adapt to any curvature therein. Stated differently, the neck portion 6016 of the handle portion 6003 has a smaller area moment of inertia when bent perpendicular to the axis 6020′ than the handle portion 6012′.

The following examples are offered to illustrate, but not to limit the herein described embodiments.

### Example 1. Sample Collection

Sample collection was performed using a standard flocked swab (e.g., from Copan) in one nostril and a swab, as described herein, in the other nostril. For reference the “T1 Ct-molded with bristles” data corresponds with the swab head illustrated on FIGS. 28-31 while the “T2 Ct—NextSwab” data corresponds with the swab head illustrated on FIGS. 35-48. RNA was isolated from the nasopharyngeal swab specimens via a QIAGEN AIAamp DSP Viral RNA Mini Kit. The RNA was reverse transcribed to cDNA and then amplified (Applied Biosystems 7500 Fast Real-Time PCR System with software version 2.3. During the amplification process, the probe anneals to a specific target sequence located between the forward and reverse primers. During the extension phase of the PCR cycle, the 5′ nuclease activity of Taq polymerase degrades the bound probe, causing a reporter dye (FAM) to separate from the quencher dye (MGBNFQ), generating a fluorescent signal. Fluorescence intensity is monitored at each PCR cycle. An internal control targeting RNase P gene is used to verify that nucleic acid is present in every sample. This also serves as the extraction control to ensure there was enough nucleic acids from each sample collected by each swab respectively.

The efficiency of sample collection between the two different swabs was measured by the detection of RNase P (RP) Ct value. A lower Ct value is indicative of better collection outcomes. Based on the Ct values below (see Table 1), the swab designed described herein (FIGS. 28-31, 35-42) performed as well or better than the standard flocked swab.

TABLE 1
Ct Values of Select Molded Swab Heads versus
Standard Flocked Swabs.

| | T1 Ct-molded | T2 Ct- | RP Ct- |
|---|---|---|---|
| | with bristles | NextSwab | Flocked |
| Individual | (FIGS. 28-31) | (FIGS. 35-42) | Swab |
| A | Undetermined | 28.39418221 | 28.83027458 |
| B | 30.53497887 | 29.40417862 | 29.90654564 |
| C | Undetermined | 27.64152718 | 27.99189377 |
| D | 5.663459301 | 26.53832054 | 26.95277786 |
| E | 25.41960144 | 24.55483437 | 25.18572235 |
| F | Undetermined | 24.87270546 | 25.3590641 |
| G | Undetermined | 25.87874413 | 26.27615929 |

Although the invention has been described in detail with reference to certain preferred embodiments, variations and modifications exist within the scope and spirit of one or more independent aspects of the invention as described.

1. A swab for collecting biological samples, the swab comprising:
a head portion; and
a handle portion extending from the head portion to define an axis therethrough, and wherein the head portion and the handle portion are formed together from a single piece of material.

2. The swab of claim 1, wherein the single piece of material includes medical-grade polypropylene.

3. The swab of claim 1, wherein the single piece of material is a non-fibrous and non-foam type material.

4. The swab of claim 1, wherein the head portion has a larger cross-sectional area than the handle portion.

5. The swab of claim 1, wherein the head portion and the handle portion are molded together during a single molding step.

6. The swab of claim 1, wherein the handle portion includes a grip portion and a neck portion extending from the grip portion, and wherein the grip portion has a greater area moment of inertia than the neck portion.

7. The swab of claim 1, wherein the head portion includes a base, a tip, and a plurality of legs extending between the base and the tip.

8. The swab of claim 7, wherein at least two legs from the plurality of legs form at least one gap therebetween.

9. The swab of claim 7, wherein the head portion includes a center leg and at least two exterior legs, and wherein each exterior leg is spaced a distance from the center leg to produce a gap therebetween.

10. The swab of claim 1, wherein the head includes at least one protrusion extending radially outwardly therefrom.

11. A swab for collecting biological samples, the swab comprising:
a handle having a first end and a second end opposite the first end; and
a head coupled to the handle at the first end thereof, wherein the head includes:
a base proximate the first end of the handle,
a tip spaced a distance from the base opposite the handle, and
a plurality of legs each extending between the base and the handle.

12. The swab of claim 11, wherein the handle and the head are formed from a single piece of material.

13. The swab of claim 11, wherein the handle and the head are formed separately.

14. The swab of claim 11, wherein the plurality of legs includes a center leg and at least two exterior legs.

15. The swab of claim 14, wherein the head defines a gap between the center leg and at least one adjacent exterior leg.

16. The swab of claim 15, wherein each gap is at least partially enclosed by the center leg, the at least one adjacent exterior leg, the base, and the tip.

17. The swab of claim 15, wherein each gap is open to the outside of the head in at least one location.

18. The swab of claim 11, wherein the head includes an exterior, and wherein the head includes at least one location on the exterior having a first roughness and at least one location on the exterior having a second roughness different than the first roughness.

19. The swab of claim 11, wherein at least one leg of the plurality of legs includes a protrusion extending therefrom.

20. A method of manufacturing a swab for collecting biological samples, wherein the swab includes a handle portion and a head portion, the method comprising:
providing a mold defining a mold cavity, wherein the mold cavity at least partially defines both the head portion and the handle portion;
injecting a first material into the mold cavity to produce both the head portion and the handle portion of the swab; and
ejecting the resulting part from the mold cavity.

21. The method of claim 20, wherein the first material includes one of a thermoplastic elastomer, polyethylene, polypropylene, and silicone.

22. The method of claim 20, wherein the mold is a two-part mold.

23. The method of claim 20, further comprising overmolding a second material onto the first material.
